(19) United States
(12) Patent Application Publication
(10) Pub. No.: US 2005/0255578 A1
(43) Pub. Date: Nov. 17, 2005

(54) Device for analyzing constituents of a sample

(76) Inventor: Bernhard Ronacher, Linz (AT)

Correspondence Address: FULBRIGHT & JAWORSKI L.L.P., 600 CONGRESS AVE., SUITE 2400, AUSTIN, TX, 78701, US

(21) Appl. No.: 10/516,049

(22) PCT Filed: May 28, 2003
(86) PCT No.: PCT/AT03/00154

§ 371 Date: Nov. 29, 2004

(30) Foreign Application Priority Data
May 29, 2002 (AT) A 831/2002

(51) Int. Cl. C12M 1/34

(52) U.S. Cl. 435/287.1

(57) ABSTRACT

The invention relates to a device for analysing constituents of a sample (5). Said device comprises sensor molecules that are deposited on one surface of a carrier, which optionally bond with the constituents to be analysed. The aim of the invention is to reliably detect even negligible quantities of sample constituents to be analysed and to obtain good results from said analysis. To achieve this, the carrier consists of a centrifuge head (1) that can be inserted into a sample container (2), leaving a radial annular gap (9) exposed. The peripheral surface (3) of the centrifuge head forms the surface that is to be provided with the sensor molecules.

### TECHNICAL FIELD

The invention relates to a device for analyzing constituents of a sample, said device comprising sensor molecules deposited on a surface of a carrier, which sensor molecules optionally bond to or interact with the constituents to be analyzed.

### PRIOR ART

Known devices work with sensor molecules applied in regions on a carrier platelet, which sensor molecules offer a binding site to constituents of a sample to be analyzed, or which interact with constituents of the sample. The sensor molecules are arranged, e.g., distributed in a certain pattern over the carrier platelet, wherein the most varying sensor molecules may be provided on the carrier platelet for the most varying constituents of the sample to be analyzed. The analysis is effected by a molecular bond of sample constituents with the sensor molecules. Whether or not certain constituents are contained in a sample thus can be recognized by whether or not such constituents have bonded with the corresponding sensor molecules. Such a molecular bond is recognized, e.g., by means of an optical, an electro-chemical measurement or by a measurement of the change in mass. Usually, such devices are called chemical sensors. If the constituents to be analyzed are biomolecules, the device is called a biosensor. If several different sensor molecules are accommodated on a carrier, this is called a biochip. Such biochips may consist of a number of biosensors arranged on a microchip, and on this microchip electric sensors may be arranged in gate form which are associated with individual sensor molecules. With these electric sensors it can be measured whether or not constituents of the sample have bonded with sensor molecules. As has already been mentioned, the most varying sensor molecules for the most varying constituents of a sample may be accommodated on a biochip and analyzed simultaneously.

In the known devices, the sample is applied to the sample platelet, and subsequently, the sample platelet usually is covered by a further platelet so as to ensure a uniform distribution of the sample over the entire carrier platelet surface, which is indispensible for exact measurements. After a desired residence time of the sample on the carrier, the sample is removed, and bonds, or interactions, respectively, between sensor molecules and constituents of the sample can be measured. However, as has already been mentioned, since the known biochips take up a great number of the most varying sensor molecules on their surface, with the known devices it may always happen that constituents present at a low concentration in the sample cannot be recognized by these devices, particularly when a sensor molecule just does not happen to be in the vicinity of a corresponding constituent to be measured. Therefore, with these known devices it is not ensured that constituents present in the sample will find their matching sensor molecule.

From WO 02/08457 A2 it is known to convey a sample along reaction areas, and for this purpose the sensor molecules are placed in the groove bottom of a screw, which screw then is screwed into an internal thread provided in a sample container. In doing so, a flow channel forms in the region between the groove bottom of the screw and the internal thread, through which flow channel the sample is conveyed. However, with such a device, constituents present in the sample at a low concentration will be caught by a sensor molecule with high probability only if the sample is conveyed several times through the flow channel.

### DESCRIPTION OF THE INVENTION

The invention is based on the task of further developing a device of the initially defined kind such that even the slightest amounts of constituents contained in a sample can be detected by it with the simplest means. At the same time, however, an automated, simultaneous analysis of as large a number of constituents as possible in a sample shall be rendered feasible.

The invention solves this problem in that the carrier consists of a rotor insertable into a sample container by leaving clear a radial annular gap, the peripheral surface of the rotor forming the surface to be provided with the sensor molecules.

Since the carrier is designed as a rotor which immerses in a sample container, a flow is forced on the sample during the rotational movement of the rotor in the sample container, as a consequence of which there is always a relative movement between the sample and the sensor molecules. By this relative movement between the sample and the sensor molecules, it is ensured that the constituents to be analyzed with very high probability will find their corresponding sensor molecules on account of the active sample transport along the reaction areas. Due to the rotational movement of the rotor, a parallel analysis of the most varying constituents of a sample in one measurement (analysis) is feasible, optionally in fully automated manner.

For example, the sample can be introduced in the sample container before insertion of the rotor, or the rotor has at least one axial or radial flow channel opening into the sample container so that the sample can be introduced into the annular gap between the sample container and the rotor, when the rotor has been inserted in the sample container. In this way, e.g., a larger amount of a sample could be pumped through the device for analysis of the former. Moreover, the sample container could have at least one associated channel for a sample transport into and out of the sample container, and when the analysis has been effected, this (these) flow channel(s) may be used for supplying and discharging cleaning agents or agents for recognizing bonds.

In order to provide the best conditions possible for the analysis, it is advantageous if the sample container, at least in regions thereof, has an associated heating and/or cooling means. In this way, e.g., the reaction temperatures for different sensor molecules can be adapted.

To ensure as simple and rapid a constituent analysis as possible, the surface of the rotor may comprise electric sensors on which the sensor molecules are placed. In this case, bonds present between sensor molecules and the constituents to be analyzed can then be concluded by measurements of voltage, resistance and/or current measurements.

The sample container may just as well have an associated optical measurement means with which the bonds may, e.g. be recognized due to a changing luminescence and/or fluorescence. These measurement means optionally serve to observe and measure the interactions which have occurred between sample molecules and sensor molecules during the incubation (without having to remove the rotor from the sample container), i.e., if required, also while a flow is forced on the sample by the rotor so that the number and type of the reactions can be recorded in dependence on the analysis time.

To further improve the sample transport along the reaction areas, i.e. the sensor molecules, directing means for a sample flow may be associated with the sample container and/or with the rotor. (For instance, a helical configuration of rotor and/or sample container).

To improve the reaction conditions, it may be advantageous to provide the rotor with surface structures on which the sensor molecules are placed. Thus, the rotor may, e.g., have elevations or depressions. The rotor may also have an associated membrane on which the sensor molecules are arranged. In this case, the membrane will be pulled over the carrier and inserted with the carrier into the sample container, before or after the sensor molecules have been applied to the membrane.

For a flawless mounting of the rotor in the sample container, it is advisable to design the rotor and/or the sample container, at least in portions thereof, with a multiple-face slide bearing.

### SHORT DESCRIPTION OF THE DRAWINGS

In the drawing, the invention is illustrated by way of a schematic exemplary embodiment. Therein,

FIG. 1 shows a sample container and an inventive rotor, and

FIG. 2 shows the sample container of FIG. 1, with the rotor inserted therein.

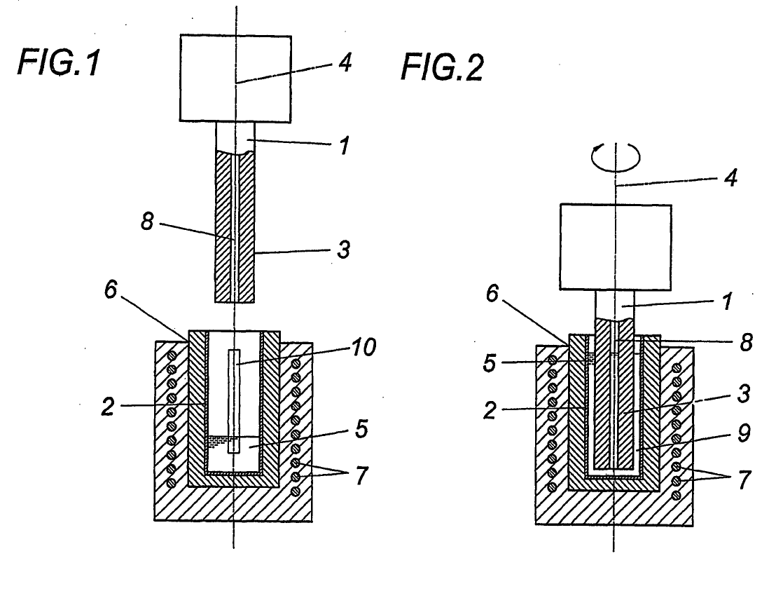

### MODE OF CARRYING OUT THE INVENTION

A device for analyzing constituents of a sample consists of a carrier designed as a rotor 1, and a sample container 2, in which the rotor 1 is insertable. According to the exemplary embodiment, the rotor 1 is of circular-cylindrical shape and has various sensor molecules not further illustrated on its peripheral surface 3, which sensor molecules optionally bond with the constituents of a sample 5 to be analyzed, or interact with the constituents, the rotor 1 being mounted so as to be rotatable about an axis of rotation 4. The sample container 2 is inserted in a holding means 6 which accommodates a heating and/or cooling means 7. The rotor 1 has a flow channel 8 opening into the sample container 2, through which flow channel the sample 5, or a cleaning agent, respectively, can be introduced into the annular gap 9 between the rotor 1 and the sample container 2. In order to allow for an immediate recognition of possible bonds, or interactions, respectively, between constituents of the sample and sensor molecules during analysis, the sample container 2 has a detection window 10 through which an optical measurement means not further illustrated can measure bonds possibly present. Likewise, the peripheral surface 3 of the rotor 1 preferably has electric sensors on which the sensor molecules are placed.

The device according to the invention is suited for an analysis of biological constituents in unknown samples, the analysis being based on interactions between known test molecules and sample constituents reacting therewith (e.g. DNA-DNA, antibody-antigen). The device according to the invention is designed for the fully automated laboratory run for routine use in diagnostics. Compared with the devices known from the prior art, the rotor 1 serving as the carrier for the sensor molecules allows for an active sample transport along the reaction areas, whereby even the slightest constituents contained in a sample can be recognized with great certainty. With the device according to the invention, an automation of the individual working steps required for analysis and an associated computer-assisted documentation of the entire analysis procedure is possible. The temperature of the sample, the residence time of the sample in the device, a possibly present gas mixture and the rotational speed of the rotor can be exactly controlled and regulated during the analysis procedure.

1-7. (canceled)

8. A device for analyzing constituents of a sample comprising:
a sample container;
a carrier comprising a rotor that is insertable into the sample container during use leaving clear a radial annular gap, a peripheral surface of the rotor forming a surface on which sensor molecules are deposited;
at least one flow channel opening into the sample container disposed in the rotor; and
a flow director adapted to direct the sample flow during use associated with the sample container and/or with the rotor.

9. The device of claim 8, wherein the sensor molecules bind to or interact with constituents of the sample during use.

10. The device of claim 8, wherein the sample container has an associated heater and/or cooler.

11. The device of claim 8, wherein the peripheral surface of the rotor comprises electric sensors on which the sensor molecules are deposited.

12. The device of claim 8, wherein an optical measurement device is associated with the sample container.

13. The device of claim 8, wherein the rotor is provided with surface structures on which the sensor molecules are deposited.

14. The device of claim 8, wherein the rotor and/or the sample container at least in portions thereof is designed as a multiple-face slide bearing.

15. The device of claim 8, wherein the rotor has an associated membrane on which the sensor molecules are arranged.
